(19) United States
(12) Patent Application Publication
(10) Pub. No.: US 2023/0212666 A1
(43) Pub. Date: Jul. 6, 2023

(54) SEGMENTATION AND LABELING FOR SINGLE MOLECULE SEQUENCING

(71) Applicant: Seagate Technology LLC, Fremont, CA (US)

(72) Inventors: Raman Venkataramani, Longmont, CO (US); William M. Radich, Berthoud, CO (US); Jason Bellorado, San Jose, CA (US)

(21) Appl. No.: 17/565,696

(22) Filed: Dec. 30, 2021

(51) Int. Cl. C12Q 1/6874; G16B 30/10

(52) U.S. Cl. CPC C12Q 1/6874; G16B 30/10

(57) ABSTRACT

Systems and methods are disclosed for performing segmentation and labeling of signals generated by single molecule sequencing. In certain embodiments, a method may comprise receiving a training signal generated by molecular detection, segmenting the training signal into a set of events, determining signal characteristics for the set of events, generating a Hidden Markov Model (HMM) based on the set of events and the signal characteristics. The HMM may also be applied to a second signal and may responsively segment the second signal into a second set of events and label the second set of events based on the signal characteristics. A labeled sequence signal output may be provided that includes the second set of events and corresponding labels generated by the HMM.

### SUMMARY

In certain embodiments, a method may comprise receiving a training signal generated by molecular detection, segmenting the training signal into events, determining signal characteristics for individual ones of the events, and fitting a Hidden Markov Model (HMI) based on the events and the signal characteristics. The method may further comprise receiving a second signal generated by molecular detection, applying the HMI to the second signal and, in response, segmenting the second signal into the events and labeling the events based on the signal characteristics of the individual ones of the events.

In certain embodiments, an apparatus may comprise a circuit configured to receive a training signal generated by molecular detection. The circuit may segment the training signal into events, determine signal characteristics for individual events, and fit a Hidden Markov Model (HMM) based on the events and the signal characteristics. The circuit may receive a second signal generated by molecular detection. The circuit may apply the HMI to the second signal and responsively segment the second signal into the events and label the events based on the signal characteristics of the individual events.

In certain embodiments, a memory device may store instructions that, when executed by a processor, cause the processor to perform a method comprising receiving a training signal generated by molecular detection, segmenting the training signal into events, determining signal characteristics for individual events, and fitting a Hidden Markov Model (HMI) based on the events and the signal characteristics. The method performed by the processor may further comprise receiving a second signal generated by molecular detection, applying the HMM to the second signal and responsively segmenting the second signal into the events and labeling the events based on the signal characteristics of the individual events.

### BRIEF DESCRIPTION OF THE DRAWINGS

FIG. 1 illustrates a system configured to generate a signal based on single molecule sequencing, in accordance with certain embodiments of the present disclosure.

FIG. 2 illustrates a system configured to perform segmentation and labeling for single molecule sequencing, in accordance with certain embodiments of the present disclosure.

FIG. 3 illustrates a training signal generated by single molecular sequencing; in accordance with certain embodiments of the present disclosure.

FIG. 4 illustrates a system configured to perform segmentation and labeling for single molecule sequencing, in accordance with certain embodiments of the present disclosure.

FIG. 5 illustrates a system configured to perform segmentation and labeling for single molecule sequencing, in accordance with certain embodiments of the present disclosure.

FIG. 6 illustrates a system configured to perform segmentation and labeling for single molecule sequencing, in accordance with certain embodiments of the present disclosure.

FIG. 7 illustrates a system to perform segmentation and labeling for single molecule sequencing, in accordance with certain embodiments of the present disclosure.

FIG. 8 illustrates a system configured to generate signals based on amino acid sequencing, in accordance with certain embodiments of the present disclose.

FIG. 9 illustrates a system configured to perform segmentation and labeling for amino acid sequencing, in accordance with certain embodiments of the present disclosure.

FIG. 10 illustrates a system configured to perform segmentation and labeling for amino acid sequencing, in accordance with certain embodiments of the present disclosure.

### DETAILED DESCRIPTION

In the following detailed description of certain embodiments, reference is made to the accompanying drawings which form a part hereof, and in which are shown by way of illustration of example embodiments. It is also to be understood that features of the embodiments and examples herein can be combined, exchanged, or removed, other embodiments may be utilized or created, and structural changes may be made without departing from the scope of the present disclosure.

In accordance with various embodiments, the methods and functions described herein may be implemented as one or more software programs running on a computer processor or controller. Dedicated hardware implementations including, but not limited to, Application Specific Integrated Circuits (ASICs), programmable logic arrays, and other hardware devices can likewise be constructed to implement the methods and functions described herein. Methods and functions may be performed by modules, which may include one or more physical components of a computing device (e.g., logic, circuits, processors, sensors, etc.) configured to perform a particular task or job, or may include instructions that, when executed, can cause a processor to perform a particular task or job, or may include any combination thereof. Further, the methods described herein may be implemented as a computer readable storage medium or memory device including instructions that, when executed, cause a processor to perform the methods.

DNA sequencing can be accomplished by various methods. Two of the first-generation sequencing methods are the Sanger dideoxy terminator base method and the Maxam-Gilbert (chemical cleavage) method. The Maxam-Gilbert method works by selectively cleaving the DNA strand at instances of a specific chosen base (A, C, G or T). The Sanger dideoxy method works by replicating a strand but stopping at a random instance of the specific base. Both methods are similar in that they generate short DNA fragments (of random lengths) terminating at an instance of the selected base. A technique called polyacrylamide gel electrophoresis can be used to measure the lengths of the resulting fragments. Polyacrylamide gel electrophoresis indicates the positions of the specific base in the original DNA strand. The process can be done separately for each of the four bases to get the complete DNA sequence. A limitation of these two methods may be that they only work well for sequences that are 100 bases long. Beyond this limit, the uncertainty in the position of each base becomes unacceptable. Longer sequences must be broken down, sequenced individually, and stitched together like pieces of a jig saw puzzle using genome assembly algorithms.

Other methods of DNA sequencing include massively parallel sequencers that are still limited to processing short fragments. The idea of “single molecule sequencing” is to avoid fragmentation of the DNA and try to sequence the entire single stranded DNA (ssDNA) molecule in a single pass. Single-molecule sequencers can sequence molecules over 10 kilobases long. Some single-molecule sequencers can use an optical waveguide system to sequence DNA, while other single molecule sequencers can use a nanopore device with a sensor to measure electrical currents.

Nanopore DNA sequencing allows for low-cost high-throughput genome sequencing. Nanopore DNA sequencers comprise a tiny pore about 2 nm wide, just wide enough for a single stranded DNA molecule (ssDNA) to pass through. As a negatively charged single stranded DNA translocates under an external electric field by a process called electrophoresis, the device sensor picks up a transverse ionic or tunneling current between two electrodes. By measuring the changes in the current, with the movement of each nucleotide through the pore, and applying a base-calling algorithm to the signals, the bases in the ssDNA sequence can be detected.

Protein sequencing is another important problem within the realm of single molecule sequencing. Here the goal is to identify the component amino acids in a protein sequence. Although the underlying sequencing technology may vary from one application to another, the fundamental signal processing problem of segmenting the measured waveforms and labeling them into the constituent parts is similar.

Although signal events in the nanopore DNA model are simple level shifts contaminated by additive noise, this model is inadequate for other applications where these signal characteristics could be much more complex than level changes alone. While nanopore DNA sequencing may produce signal events with different signal levels, sequencing other molecule types like protein sequences may produce different events that do not have noticeably different signal levels. Instead, protein sequencing events may comprise different noise profiles such a correlated non-Gaussian noise, and even spiky noise. Some nanopore DNA models are unable to effectively segment and label signals that lack noticeably different signal levels.

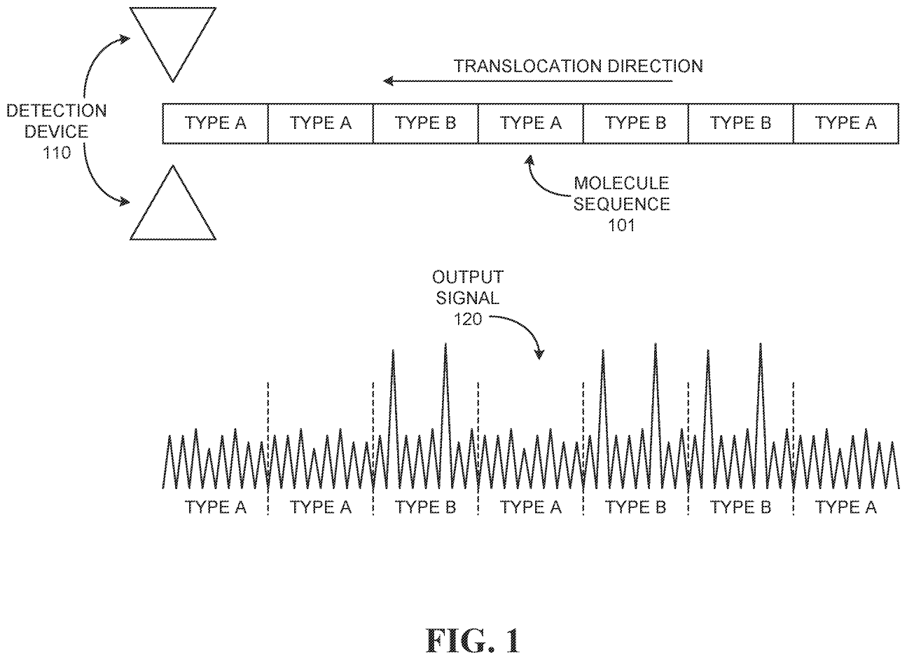

FIG. 1 illustrates a system 100 configured to generate a signal based on molecular sequencing for single molecule segmentation and labeling, in accordance with certain embodiments of the present disclosure. System 100 may include molecule sequence 101, detection device 110, and output signal 120. Molecule sequence 101 can comprise a polypeptide, an amino acid sequence, a Single Stranded Deoxyribonucleic Acid (ssDNA) sequence, a double stranded DNA sequence, a Ribonucleic Acid (RNA) sequence, or some other type of molecule sequence that comprises different types of monomers. Detection device 110 may comprise a nanopore sensor, a fixed gap detection device, or some other type of device configured to detect a molecule sequence. In some examples, molecule sequence 101 may include a sequence of nucleotide bases from the set including adenine (A), cytosine (C), guanine (G), and thymine (T). In some examples, molecule sequence 101 may include a sequence of amino acids like Glycine (GLY), Tyrosine (TYR), Histidine (HIS), and the like that are chemically bound to one another to form a polypeptide. Output signal 120 is an example of a readout signal generated by the translocation of molecule sequence 101 through detection device 110. Output signal 120 comprises a waveform with different types of signal characteristics that correspond to the molecules that comprise molecule sequence 101. In this example, molecule sequence 101 can be comprised of “type-A” and “type-B” molecules and output signal 120 comprises a simulated sequencer output.

Detection device 110 may interact with molecule sequence 101 to detect changes in molecule sequence 101 that indicate the timing of events. An “event” comprises the movement of a single molecule of molecule sequence 101 through detection device 110. For example, by measuring changes in current with the passing of molecule sequence 101 through detection device 110, detection device 110 can detect the constituent molecule types in molecule sequence 110.

As molecule sequence 101 passes through the detection device 110 in the translocation direction indicated on FIG. 1, the type A constituent molecule can be sampled. Detection device 110 may generate a current as molecule sequence 101 passes through, and sample values are taken from the current. However, the method detection device 110 uses to interact with molecule sequence 101 is not limited. The samples may be taken continuously, periodically, randomly, or may be taken over some other type of time interval. The sample values may be indicated by the solid lines that comprise output signal 120. Detection device 110 may sample a single molecule multiple times before the molecule completely passes through detection device 110. The process may continue as the type A and type B constituent molecules pass through the detection device 110. Detection device 110 can generate the output signal 120 that indicates the interactions between the single molecules and the detection device. A sequence of different waveforms that correspond to type A and type B constituent molecules may be determined based on the samples. The different waveforms of output signal 120 may be used to identify individual events and to identify the molecule types that correspond to each.

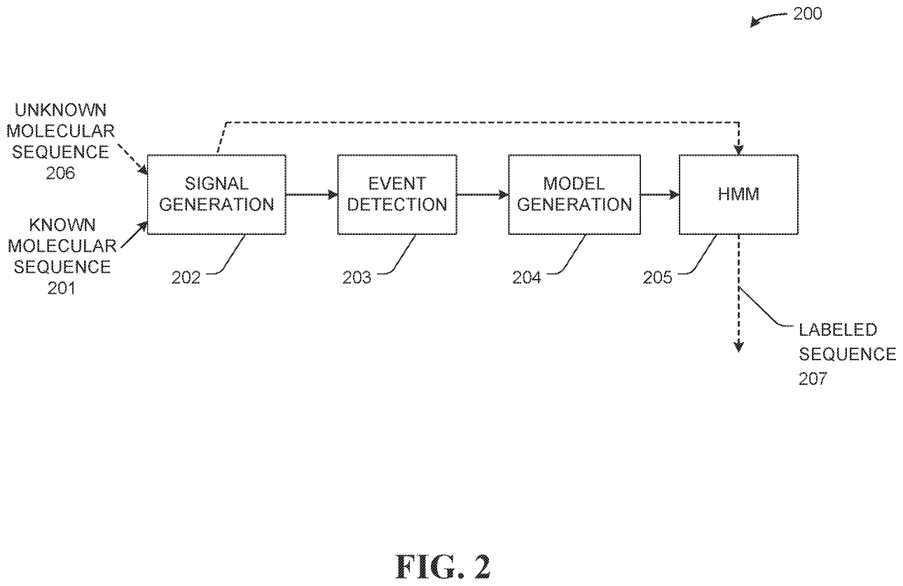

FIG. 2 is a diagram of a system, generally designated 200, configured to generate an HMM module for segmentation and labeling in single molecule sequencing, in accordance with certain embodiments of the present disclosure. In particular, FIG. 2 depicts an example Hidden Markov Model (HMI) generation system, including a number of processing blocks, modules, or system components configured to perform signal generation, event detection, and model generation. System 200 can include known molecular sequence 201, signal generation module 202, event detection module 203, model generation module 204, HMM module 205, unknown molecular sequence 206, and labeled sequence 207. The processing blocks may be included on or executed by one or more modules or physical components of a signal processing channel circuit. In some examples, signal generation module 202 may correspond to detection device 110 illustrated in FIG. 1. In some examples, system 200 may include additional processing blocks that are not illustrated in FIG. 2.

Signal generation module 202 can receive a known molecular sequence 201. The known molecular sequence 201 can comprise a molecular strand with known constituent molecules arranged in a known sequence. For example, known molecular sequence 201 may comprise a polypeptide chain with a known sequence of amino acids (e.g., GLY-GLY-HIS-HIS-GLY-GLY-TYR). Signal generation module 202 can generate a signal that characterizes the known molecule sequence. The signal may comprise a waveform, noise profile, other signal characteristic, or a combination thereof that indicates individual molecules that comprise the known molecular sequence 201. The signal generation module 202 can convert the analog signal into a digital format and transfer it to event detection module 203.

Event detection module 203 can segment the signal into events based on the known constituent molecules and the known sequence of sequence 201. An event represents the passing of one molecule of known molecular sequence 201 through a molecular sequencer. For example, event detection module 203 may segment the signal in a manner similar to output signal 120 illustrated by FIG. 1. Event detection module 203 can label the events based on known molecule sequence 201. For example, known sequence 201 may comprise a DNA sequence of A-C-T-G-G-T-A-C and event detection module 203 may label the events accordingly. Event detection module 203 can transfer the segmented and labeled signal to model generation module 204.

Model generation module 204 can generate an HMM to characterize the signal characteristics associated with each event. An HMI is an artificial statistical model that utilizes observed parameters (e.g., observable sequential symbols) to identify hidden parameters, which can then be used for further determinations of properties of other signals. In system 200, each HMI can correspond to an event type and each event type can correspond to a set of signal characteristics unique to that event type. For example, model generation module 204 can fit a probabilistic model for the HMI, where each node in the HMM represents an individual signal characteristic, and the edges indicate the probability a first signal characteristic will transition to another (or the same) signal characteristic. Model generation module 204 may also determine the transition probabilities for the edges. For example, model generation module 204 may implement a Baum-Welch algorithm to learn the emission and state transition probabilities to generate the HMI. Model generation module 204 can repeat this process until the signal characteristics for each event type have been classified. Model generation module 204 may combine individually generated HMMs into an overall HMI at the HMM module 205.

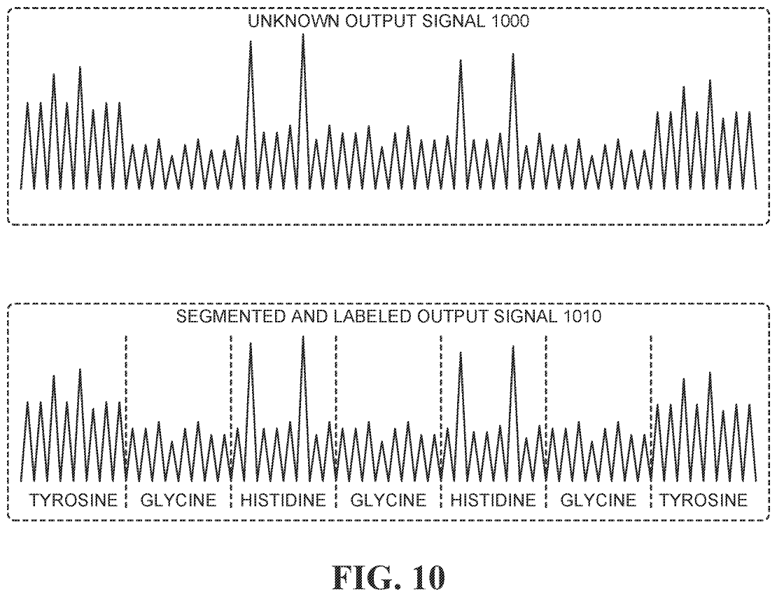

Signal generation module 202 can also receive an unknown molecule sequence 206. An unknown molecular sequence 206 may comprise the same types of molecules that comprise known molecular sequence 201 but in an unknown arrangement. Signal generation module 202 can generate an analog signal representative of unknown molecule sequence 206, convert the analog signal to a digital representation of the analog signal, and transfer the digital signal to HMM 205. HMI module 205 can then segment the digital signal into events. HMI module 205 may determine the probability that the observed signal characteristics correspond to a molecule type. For example, HMM module 205 can label the events based on the probability that their signal characteristics correspond to the molecule type. For example, HMM module 205 may estimate a probability that the signal characteristics of an event is the nucleobase adenine and responsively label the event as adenine based on the probability. Once the signal has been labeled, HMM module 205 can emit a labeled sequence 207. An example of a labeled output sequence or signal is provided in FIG. 10.

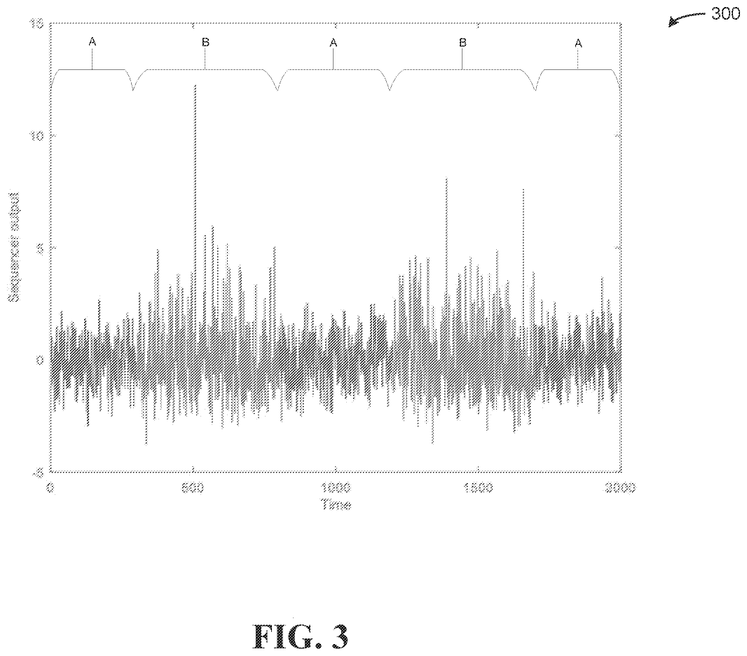

FIG. 3 illustrates a chart 300 of a training signal generated by single molecule detection of a known molecular sequence, in accordance with certain embodiments of the present disclosure. A simulated example output sequence/signal comprises five signal events that correspond to either a type A or type B molecule. Signal events of type A may comprise Gaussian noise and signal events of type B may comprise spiky noise. The y-axis of chart 300 charts a magnitude of a simulated sequencer output, such as in a range of −5 to 15 units (such as nanoamps if the measurement is an electrical current), however other values may be used. The x-axis of chart 300 charts time, such as in a range of 0 to 2000 clock cycles, however other values may be used. In some examples, the training signal can be used to train an HMM to segment and label signals generated by single molecule detection.

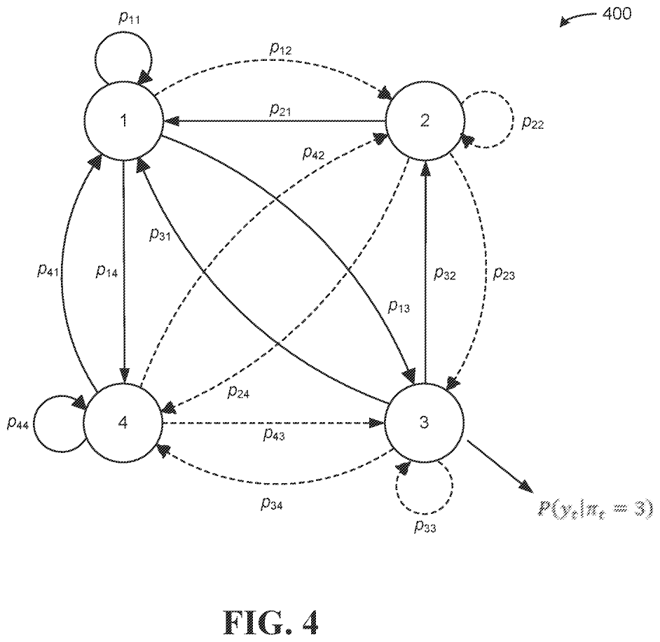

FIG. 4 illustrates a diagram of a system, generally designated 400, configured to perform segmentation and labeling of a single molecule detection signal, in accordance with certain embodiments of the present disclosure. System 400 comprises an example of a Hidden Markov Model (HMI) 400 for segmentation and labeling of a signal into separate events based on the noise characteristics of the samples.

The entire HMM 400 can represent a distinct signal characteristic for an event. The individual states of 400 may generate signals as simple as level shifts but the entire combination of HMI states can model signals with correlated samples, including spiky or other complicated behavior. For example, HMI 400 may be generated to characterize event type A depicted in chart 300 and the nodes 1-4 of HMI 400 may correspond to distinct signal levels that comprise the signal characteristics for event type A. States 1-4 are bridged by edges depicted by arrows. An edge from a state i to another state j≠i represents the end of a first signal characteristic and the beginning of a new signal characteristic. Some of the self-loops (an edge from a node to itself) may represent the continuation of the same signal characteristic from the same event and other ones of the self-loops may represent a transition to a new event with the same signal characteristic.

In general, the individual transition probabilities pᵢⱼ from state i to state j (e.g., the transition from state 1 to state 2 is labeled as p₁₂ in HMI 400) can be modeled. HMI 400 models the overall signal characteristics for a signal event generated by single molecule sequencing. In some examples, HMM 400 may accurately model the signal characteristics to be able to generate a new (idealized estimate) signal(s) to mimic a real sequencer output. The idealized signal(s) may be compared to a signal generated by single molecule sequencing to characterize the signal. HMI 400 may model a wide range of signal behaviors including correlated noise, non-Gaussian noise, spiky noise, and the like.

For explanatory purposes, let Y=y₁^{N} denote a length-N signal that a molecular sequencer outputs. For example, the length-N signal may comprise the training signal illustrated in FIG. 3. HMI 400 can model the probability density function (PDF) of the continuous valued output Y at time t. HMM 400 comprises an example of such an HMM with four states. Although HMI 400 comprises four states, in some examples HMI 400 may comprise other number of states, including many more states. In some examples, HMM 400 may comprise 128 states.

In the example HMM 400, at each time t, a state transition occurs from a state πₜ₋₁ to another state πₜ with probability P (πₜ=v|πₜ₋ₐ=u)=Pᵤᵥ. For example, the probability of transitioning from state 1 to state 2 in HMM 400 would be P(πₜ=2|πₜ₋₁=1)=P₁₂. The destination state πₜ emits an output sample yₜ with a probability P(yₜ|πₜ). In relation to the output signal, each state πₜ indicates a signal characteristic in line with the aforementioned probability. These transitions form a PDF that could be a conditional Gaussian, parametrized by its mean and variance, or any other suitable continuous probability distribution. The joint PDF of the sequence output Y and the hidden state sequence Π=π₁^{N} has the factor form:

$$P ( Y , Π ) = P ( {π}_{0} ) {\prod }_{t = 1}^{N} P ( {π}_{t} \vert {π}_{t - 1} ) P ( {y}_{t} \vert {π}_{t} )$$

In some examples, the Baum-Welch algorithm is used to learn the emission and state transition probabilities. The Baum-Welch algorithm comprises a special case of an Expectation Maximization (EM) algorithm used to find the unknown parameters of an HMM. This algorithm tries to maximize the likelihood of the observed data P(Y) over all the HMI parameters. The Baum-Welch algorithm may be used to learn the adjacency structure of HMI 400. HMM 400 starts fully connected (e.g., the solid arrows) and the algorithm prunes the edges with vanishingly small transition probabilities, say less than 10⁻⁹, in each iteration. Once the transition probability given by an edge falls below a threshold, the algorithm prunes the edge. With respect to HMI 400, the pruned edges comprise the dashed arrows. In some examples, HMM 400 comprises far fewer edges with sparse connectivity once the pruning process is complete. HMI 400 may be trained to model the sequencer output for single molecule sequencing. Specifically, HMM 400 may be used to determine the PDF P(Y, Π) for typical sequencer output signals. In some examples, the distribution generated by HMI 400 can be sampled to generate idealized signals Y that mimic those of a real sequencer.

In addition to generating signals, HMI 400 may also be used to test whether a given signal Y=y₁^{N} of unknown origin may be a typical output of the sequencer using a “likelihood test”. A normalized log-likelihood function

$$L = \frac{1}{N} log [ P ( Y ) ]$$

may be computed with respect to the estimated PDF. If a given signal scores score sufficiently high, HMM 400 may label the signal. A signal that is not a typical sequencer output would score low on this test. A suitable threshold for the score can be determined by testing on several signals from both inside and outside the class of typical sequencer outputs.

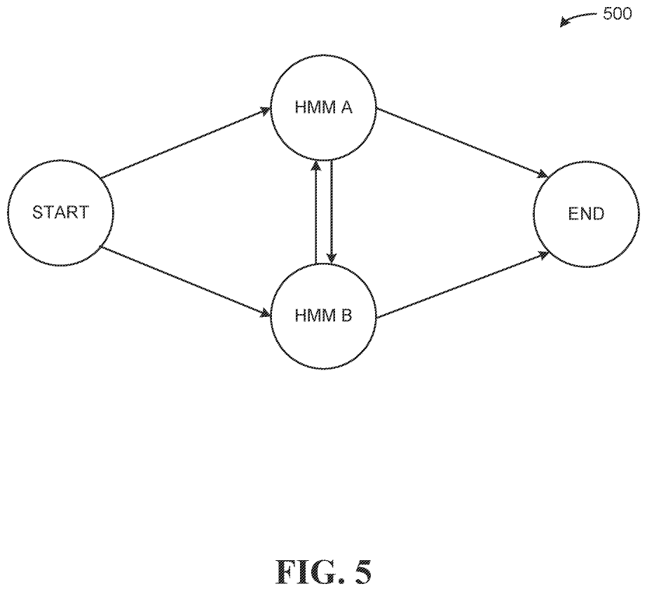

FIG. 5 illustrates a diagram of a system, generally designated 500, configured to perform segmentation and labeling of a single molecule detection signal, in accordance with certain embodiments of the present disclosure. System 500 comprises an example of an HMI module for segmentation and labeling of signal samples into separate events based on the signal noise characteristics of the samples.

In this example, HMI 500 is configured to segment and label a signal generated by single molecule sequencing for two molecule types, type A and type B. HMI 500 is configured to automatically segment a signal into the individual type A and B events. HMI 500 may be configured to segment and label the output signal illustrated in FIG. 3. When there are only two activators type-A and type-B, in the biological sample, there will be two types of signal events corresponding to these activators. A training signal with a known sequence can be used to individually generate HMM-A and HMM-B using processes similar to those described with respect to HMI 400 or elsewhere herein. For example, if the training signals are properly segmented and labeled, type-A and type-B signals can be extracted and used to separately train HMI A and HMI B for these signal types. Once HMM A and HMI B have been generated, they can be combined into HMI 500. HMM 500 may comprise all the states of HMM A and HMM B and, in some examples, HMM 500 may also include start and stop states. HMM A and HMM B can comprise internal edges (not shown) to effectively identify event types A and B respectively. HMM 500 can also comprise edges between states in HMM A and HMI B that represent event boundaries. In some examples, the transition probabilities for the event boundary edges may be set to match to the actual event transition probabilities.

Once HMM 500 is constructed, HMM 500 may perform automatic segmentation of a sequencer output Y that comprises an unknown molecular sequence of molecules type-A and type-B into events of type-A and type-B. The Viterbi algorithm may be applied to HMI 500 with a negative log-likelihood branch metric to perform the automatic segmentation. The Viterbi algorithm comprises a dynamic programming algorithm for obtaining the maximum a posteriori probability estimate of the most likely sequence of hidden states. In this case, the sequence of hidden states comprises the sequencer output Y. It should be noted that the log-likelihood metric can be used to discriminate between signals that came from different sources. Here the log-likelihood metric can be used to discriminate between the signals of type-A and type-B. The Viterbi algorithm computes the maximum-likelihood (ML) path in HMM 500 which results in the ML segmentation of the signal into type-A and type-B events.

Advantageously, HMM 500 may automatically segment and label a signal generated by signal molecule sequencing. Moreover, HMM 500 may process signals that comprise different noise profiles that correspond to different types of molecules.

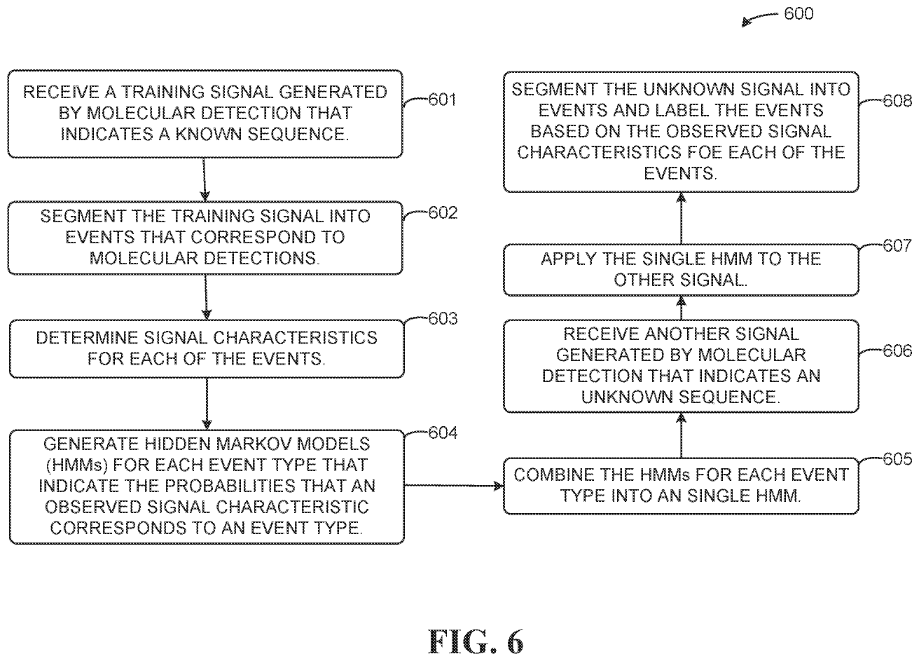

FIG. 6 illustrates a method 600 to generate an HMM to segment and label an output signal generated by single molecule detection. The method 600 can be utilized with the systems and structures provided herein. Variations of the method 600 may differ in other examples. The method 600 can include, at a receiver circuit, receiving a training signal generated by molecular detection that indicates a known molecule sequence, at 601. The method 600 may then, such as via a processing circuit, segment and label the training signal into events that correspond to molecular detections based on a known sequence of the training signal, at 602. The method 600 can determine signal characteristics for each of the events, at 603, and generate a HMM for each event type that indicates the probabilities that an observed signal characteristic corresponds to an event type, at 604. The method 600 can combine multiple HMIs for each event type into a single HMM, at 605. The method 600 can receive another signal, such as via the same receiver circuit or a different receiver circuit, generated by molecular detection that indicates an unknown sequence, at 606. The method 600 can then apply the single HMM to the other signal, at 607. The method 600 can utilize the single HMM to segment the unknown signal into events and label the events based on the observed signal characteristics for each of the events, at 608.

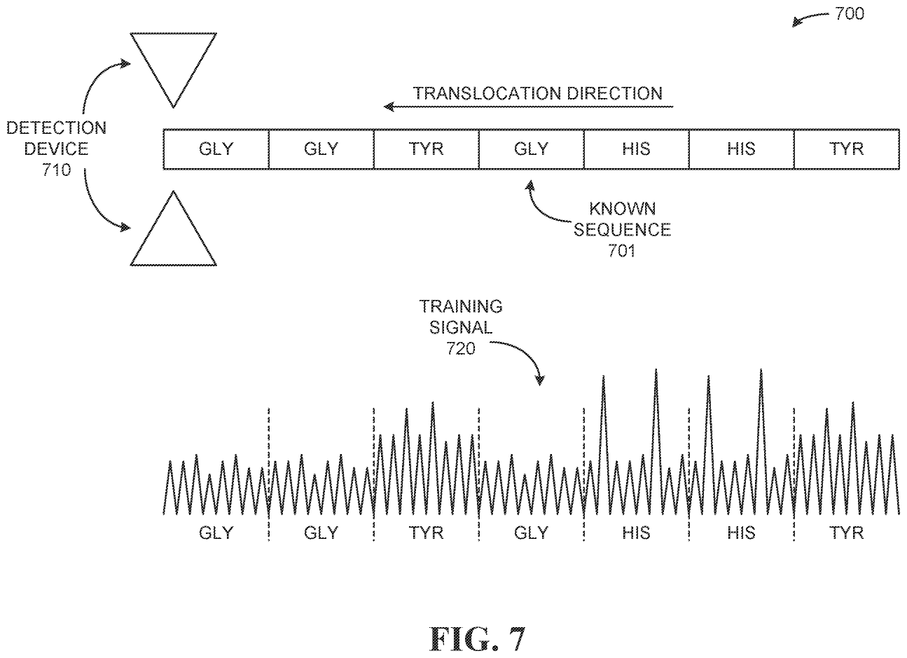

FIG. 7 illustrates a system 700 configured to generate a signal based on molecular sequencing for single molecule segmentation and labeling, in accordance with certain embodiments of the present disclosure. System 700 may include known sequence 701, detection device 710, and training signal 720. Molecule sequence 701 can comprise an amino acid chain with known molecule types and a known molecule sequence. In this example, an amino acid chain can include Glycine (GLY), Tyrosine (TYR), and Histidine (HIS) arranged in the sequence GLY-GLY-TYR-GLY-HIS-HIS-TYR. In other examples, the known sequence 701 may comprise different types and a different number of constituent molecules. Note that the number of constituent molecules is not limited. Detection device 710 may comprise a fixed gap detection device or some other type of device configured to detect an amino acid sequence.

Known sequence 701 may be translocated through detection device 710. As known sequence 701 passes through detection device 710, detection device 710 can interact with the molecule sequence 701 to detect changes in known sequence 701 that indicate the timing of events. An event may be the movement of a single amino acid through detection device 710. As the amino acids of known sequence 701 pass through and interact with detection device 710, detection device 710 can generate a training signal 720 that indicates the interactions between the amino acids and the detection device.

Training signal 720 comprises the readout generated by detection device 710. Training signal 720 can be divided into seven distinct events based on the known sequence 701. The seven distinct events may correspond to the individual amino acids that comprise the known sequence 701 and can be labeled accordingly. In some examples, training signal 720 can be used to generate and train an HMM to sequence an unknown molecular sequence. Such a resulting HMM can be used to segment and label unknown signals that comprise the same molecules as training signal 720. For example, the resulting HMM may be unable to sequence an unknown sequence comprising different molecule types like DNA bases or different types of amino acids than the ones of known sequence 701.

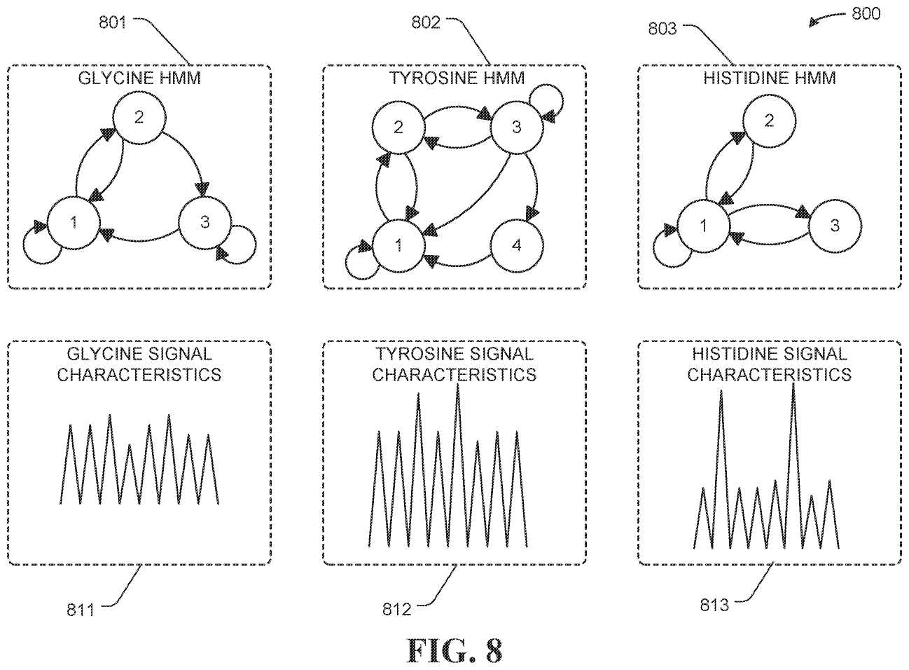

FIG. 8 illustrates a diagram of a system, generally designated 800, configured to perform segmentation and labeling of a signal generated by single molecule detection, in accordance with certain embodiments of the present disclosure. System 800 comprises an example of a glycine HMM 801, a tyrosine HMM 802, and a histidine HMM 803 that can be used to segment and label a signal generated by single molecular signaling. System 800 can additionally comprise signal samples 811-813 that may indicate the signal characteristics for glycine, tyrosine, and histidine. In some examples, signal samples 811-813 may be taken from a training signal, such as training signal 720, to generate an HMM for sequencing unknown sequences that comprise the amino acids glycine, tyrosine, and histidine.

In some embodiments, to train the HMIs 801-803, a molecular training sequence (e.g., sequence 701) can be modeled along X=x₁^{N} to denote the sequence of activator labels of amino acids. The signal output (e.g., training signal 720) is modeled along Y=y₁^{N} to denote the sequencer output. For example, x₁₋₇^{N} may correspond to glycine, x₈₋₁₅^{N} may correspond to glycine, and so on. In this example, the set xₜ is a sequence of discrete labels for glycine, tyrosine, and histidine and the label xₜ is attached to every signal sample yₜ. The labels xₜ can repeat themselves several times and change only at the event boundaries since an event duration can last many time steps. The HMMs 801-803 can be generated and trained using their corresponding signal samples. For each HMM, the sequences X and Y are jointly modeled. Thus, the state πₜ of HMMS 801-803 emit a new output pair (xₜ, yₜ) with a probability P(xₜ,yₜ|πₜ)=P(xₜ|πₜ)P(yₜ|xₜ,πₜ). The distribution P(xₜ|πₜ) is a discrete Probability Mass Function (PMF) that can be stored in a look-up table. The second term P(yₜ|xₜ,πₜ) is a Probability Density Function (PDF) that could be a conditional Gaussian, parametrized by it mean and variance, or some other parametrized continuous distribution. The full joint PDF of sequences X, Y, and Π has the factor form:

$$P ( X , Y , Π ) = P ( {π}_{0} ) {\prod }_{t = 1}^{N} P ( {π}_{t} \vert {π}_{t - 1} ) P ( {y}_{t} \vert {π}_{t} ) P ( {y}_{t} \vert {x}_{t} , {π}_{t} )$$

Note that the label xₜ is treated as an output of the HMM rather than a description of the state πₜ itself. By adopting this approach there is no preconceived notion of what states should represent. The Baum-Welch algorithm can be applied to HMMs 801-803 to learn the emission and state transition probabilities. This algorithm maximizes the likelihood of the observed data P(x₁^{N},y₁^{N}) over all the HMM parameters. In a similar manner to HMM 400, the adjacency structure is learned by pruning edges with vanishingly small probabilities. FIG. 8 illustrates the HMMs 801-803 after they have been pruned, however the HMMs 801-803 can start off with many more edges. In practice, the learned model for P(xₜ|πₜ) is heavily “polarized” in that the probability is very close to 0 or 1 for each of the nodes. For example, the nodes for the Glycine HMM 801 may only output the activator label for glycine. The HMM learning algorithm can learn a joint probabilistic description P (X, Y, Π) of the activator sequence X and the sequencer output signal Y. As such, the individual nodes of the HMMs 801-803 may output both a signal characteristic and an activator label.

The generative HMMs described in these examples contain states πₜ that emit a single output a pair of outputs (xₜ, yₜ). In further examples, the edges (πₜ₋₁,πₜ) in HMMs may emit the output(s), specifically P(xₜ,yₜ|πₜ₋₁,πₜ). This class of PDF is indeed a generalization of the original PDF which can be obtained turning off its dependence on the state πₜ₋₁. All the algorithms presented for the “state-emitting” formulation can be adapted equally well to the “edge emitting” case.

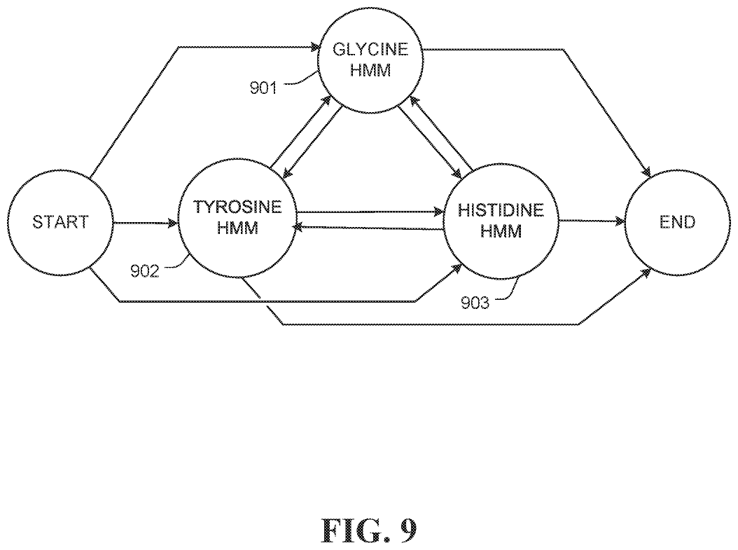

FIG. 9 illustrates a diagram of a system, generally designated 900, configured to perform automatic segmentation and labeling of a single molecule detection signal, in accordance with certain embodiments of the present disclosure. System 900 comprises an example of a HMI 900 for segmentation and labeling of an unknown signal comprising glycine, tyrosine, and histidine. The HMM 900 comprises a glycine HMM 901, a tyrosine HMM 902, and a histidine HMI 903, as well as start and stop states. In some examples, the HMMs 901-903 can correspond to the HMMs 801-803 illustrated in FIG. 8. The HMM 900 may comprise all the states of HMIs 901-903. The edges between the HMMs 901-903 can represent event boundaries, and their transition probabilities can be set to match to the actual event transition probabilities. The HMI 900 may be fed a signal generated by the single molecule sequencing of an amino acid sequence comprising glycine, histidine, and tyrosine. Upon being fed the signal, the HMI 900 can segment the signal into events and can label the events based on the probabilities indicated by the constituent HMMs 901-903.

FIG. 10 illustrates an example of the segmentation and labeling of unknown output signal 1000 generated by a system configured to generate a single molecule sequencing of glycine, histidine, and tyrosine into segmented and labeled output signal 1010.

An unknown output signal 1000 can be fed to a trained HMI (e.g., HMI 900) that is configured to process unknown signals generated by single molecule sequencing of protein chains that comprise glycine, histidine, and tyrosine. However, when the unknown signal 1000 is generated by a molecule sequence that comprises different types of molecules, the HMI can be trained to handle those molecule types. In some examples, to segment and label an unknown signal, let Π=π₁^{N} denote the hidden state sequence. The hidden state sequence denotes the sequence of nodes in the trained HMM that correctly output the observed signal characteristics of an unknown output signal 1000. To identify the correct events, the activator sequence X that maximizes the following is found by:

$$P ( X \vert Y ) = {\sum }_{Π} P ( X , Π \vert Y )$$

The summation is carried out in a manner like the marginalization technique used in the forward algorithm for HMMs so to output P(xₜ|Y) at each time t. The HMM can implement the summation and responsively segment and label output signal 1010.

In other examples, the HMM may implement the Viterbi algorithm to segment and label signal 1000. In doing so, the activator labels X can be treated as unknown or “missing data” and the Viterbi algorithm can be run on the HMM to detect the Maximum Likelihood (ML) state and activator sequences (Π, X) that explains the observed sequencer output Y.

In this disclosure, the problem of segmenting and labeling a sequencer output is addressed. There are several promising new technologies for single molecule sequencing for low-cost high throughput sequencing for medical applications as well as applications to DNA based data storage. For example, the systems and methods disclosed herein could be utilized in conjunction with the system and methods described in co-owned patent application Ser. No. 16/175,223, filed Oct. 30, 2018, entitled “Event Timing Detection for DNA Sequencing”, currently pending, the contents of which is hereby incorporated by reference in its entirety.

While some single molecule sequencing techniques produce signal events with different signal levels, sequencing other molecule types like protein sequences may produce waveforms that do not have noticeably different signal levels. Traditional signal classification methods are unable to effectively and effectively segment and label signals that lack noticeably different signal levels.

This disclosure addresses systems and methods of joint event segmentation and labeling using generative Hidden Markov Model (HMMs). Generative models are useful for generating signals that mimic a given class of training signals. The generative HMIs are used to identify the signal class from which a test signal originated. This ability to discriminate between different signal classes can be used to build a system for automatic signal segmentation and labeling. This disclosure also demonstrates a direct approach of building a generative HMM to jointly model both the labels and signal samples. Similarly, this HMM can also be used to perform automatic labeling and segmentation of sequencer signals.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Moreover, although specific embodiments have been illustrated and described herein, it should be appreciated that any subsequent arrangement designed to achieve the same or similar purpose may be substituted for the specific embodiments shown.

This disclosure is intended to cover any and all subsequent adaptations or variations of various embodiments. Combinations of the above embodiments, and other embodiments not specifically described herein, will be apparent to those of skill in the art upon reviewing the description. Additionally, the illustrations are merely representational and may not be drawn to scale. Certain proportions within the illustrations may be exaggerated, while other proportions may be reduced. Accordingly, the disclosure and the figures are to be regarded as illustrative and not restrictive.

What is claimed is:

1. A method comprising:
receiving a training signal generated by molecular detection;
segmenting the training signal into a set of events, determining signal characteristics for individual events, and generating a Hidden Markov Model (HMI) based on the set of events and the signal characteristics;
receiving a second signal generated by molecular detection;
applying the HMI to the second signal to automatically segment and label the second signal to generate an output signal; and
providing the output signal having a sequence corresponding to a set of events and corresponding labels generated by the HMM.

2. The method of claim 1 further comprising determining the signal characteristics for the set of events includes determining probabilities that the signal characteristics correspond to the set of events.

3. The method of claim 1 further comprising generating the HMI based on the set of events and the signal characteristics includes assigning probabilities to each HMM node and edge that model the node output to predict when the signal characteristics correspond to an event and transitioning to another HMI node when a node does not correspond.

4. The method of claim 1 further comprising automatically segmenting the second signal includes applying a Viterbi algorithm to the HMM and responsively segmenting the second signal into a second set of events.

5. The method of claim 4 further comprising labeling the second set of events based on the signal characteristics by estimating a likelihood that the signal characteristics correspond to the second set of events based on probabilities indicated by the HMM.

6. The method of claim 1 further comprising the signal characteristics include signal noise selected from the group of correlated noise, non-Gaussian noise, and spiky noise

7. The method of claim 1 further comprising the training signal includes a known amino acid sequence and the second signal includes an unknown amino acid sequence.

8. An apparatus comprising:
a receiver circuit configured to receive a training signal generated by molecular detection;
a processing circuit configured to segment the training signal into a set of events, determine signal characteristics for individual events from the set of events, and fit a Hidden Markov Model (HMM) based on the set of events and the signal characteristics;
the receiver circuit further configured to receive a second signal generated by molecular detection;
the processing circuit further configured to generate a labeled sequence signal by applying the HMM to the second signal and responsively segmenting the second signal into a second set of events and label the second set of events based on the signal characteristics of individual events from the second set of events; and
an output configured to provide the labeled sequence signal including the second set of events and corresponding labels generated by the HMM.

9. The apparatus of claim 8 comprising the processing circuit further configured to determine probabilities that a signal characteristic corresponds to an individual event.

10. The apparatus of claim 8 comprising the processing circuit further configured to assign probabilities to each HMM node and edge that model the node output to predict when the signal characteristics correspond to an event and transitioning to another HMM node when a node does not correspond.

11. The apparatus of claim 8 comprising the processing circuit further configured to apply a Viterbi algorithm to the HMM and responsively segment the second signal into the second set of events.

12. The apparatus of claim 8 comprising the processing circuit further configured to estimate a likelihood that an individual signal characteristic corresponds to an individual event of the second set of events based on probabilities indicated by the HMM.

13. The apparatus of claim 8 further comprising the signal characteristics include signal noise that is correlated noise, non-Gaussian noise, or spiky noise.

14. The apparatus of claim 8 further comprising the training signal includes a known amino acid sequence and the second signal includes an unknown amino acid sequence.

15. A memory device storing instructions that, when executed by a processor, cause the processor to perform a method comprising:
receiving a training signal generated by molecular detection;
segmenting the training signal into a set of events, determining signal characteristics for the set of events, and generating a Hidden Markov Model (HMM) based on the set of events and the signal characteristics;
receiving a second signal generated by molecular detection;
applying the HMM to the second signal and responsively segmenting the second signal into a second set of events and labeling the second set of events based on the signal characteristics; and
providing an output signal having a labeled sequence including the second set of events and corresponding labels generated by the HMM.

16. The memory device of claim 15 further comprising determining the signal characteristics includes determining probabilities that an individual signal characteristic corresponds to an event.

17. The memory device of claim 16 further comprising generating the HMM based on the set of events and the signal characteristics includes assigning probabilities to each HMM nodes that predict when the signal characteristics correspond to the events.

18. The memory device of claim 17 further comprising segmenting the second signal into the second set of events includes applying a Viterbi algorithm to the HMM and responsively segmenting the second signal into the second set of events.

19. The memory device of claim 18 further comprising labeling the second set of events based on the signal characteristics includes estimating a likelihood that a signal characteristic corresponds to an event of the second set events based on probabilities indicated by the HMM.

20. The memory device of claim 15 wherein the training signal corresponds to a known amino acid sequence, and the second signal corresponds to an unknown amino acid sequence.
